(19) United States
(12) Patent Application Publication
(10) Pub. No.: US 2011/0111504 A1
(43) Pub. Date: May 12, 2011

(54) BIOREACTOR AND METHOD FOR CULTIVATING CELLS AND TISSUES

(75) Inventors: Guenther Knebel, Nuertingen (DE); Sven Muehlfriedel, Frickenhausen (DE)

(73) Assignee: Greiner Bio - One GmbH, Frickenhausen (DE)

(21) Appl. No.: 12/736,866

(22) PCT Filed: May 25, 2009
(86) PCT No.: PCT/EP2009/003682

§ 371 Date: Nov. 18, 2010

(30) Foreign Application Priority Data
May 23, 2008 (AT) A 823/2008

(51) Int. Cl. C12N 5/071; C12M 3/00; C12M 1/00

(52) U.S. Cl. 435/395; 435/305.1; 435/305.2; 435/305.3; 435/305.4; 435/304.1; 435/287.1; 435/283.1; 435/293.1

(57) ABSTRACT

The invention relates to a bioreactor (1) for cell and tissue culture for the mechanical stimulation and for perfusion of tissue or cell cultures, comprising an upper part (3) and a lower part (4) which is connected to said upper part (4) and which comprises a tissue culture chamber (8), wherein at least one distributor plate (12) is arranged in the tissue culture chamber (8), and a method for cultivating cells and tissues in the bioreactor (1).

The invention relates to a bioreactor for cell and tissue culture for the mechanical stimulation and/or perfusion of tissue or cell cultures, comprising an upper part and a lower part connected thereto with a tissue culture chamber, and a distributor plate for cell and tissue cultures and a method for cultivating cells and/or tissues in a bioreactor.

For a plurality of experimental studies, such as e.g. corrosion studies on reconstructed skin, metabolism studies on reconstructed liver, and therapy methods, such as e.g. cartilage transplantation after trauma to the knee cartilage, reconstructed tissue is used. The aim of tissue reconstruction is to create functional tissue from individual cells, whereby the tissue properties should be as close as possible to those in the living organism. It is important in this case to maintain the level of differentiation of the cells, the presence of several cell layers and an extracellular matrix. For the tissue reconstruction petri dishes, multiwall plates, cell culture inserts and bioreactors are used.

Autologous cartilage transplantation (ACT) is a method for treating cartilage injuries. The main application of ACT is the treatment of cartilage defects in the knee joint, which may be caused by sports accidents. In this case in a biopsy donor-suitable (autologous) cartilage cells are removed, partly with the use of a carrier material (e.g.: collagen I-gel), cultivated ex vivo and reimplanted in the donor for repair to the cartilage damage. During the ex vivo-cultivation the cartilage cells can be propagated, whereby the increased number of cells necessary for the repair of large-area cartilage damage is made available. A generally known problem is the dedifferentiation of the cartilage cells, which is associated with the admission to cell propagation. After reimplantation dedifferentiated cartilage cells can form atypical cartilage. An example of this is the formation of fibre cartilage after the implantation of dedifferentiated cartilage cells into damaged parts of the knee joint. Unlike typical knee hyaline cartilage fibre cartilage only withstands the high pressure in the knee joint for a short period. The result is the premature failure of the implant.

Methods are being researched by means of which dedifferentiated cartilage cells can still be redifferentiated prior to the implantation. In addition to the use of three-dimensional carrier materials cultivation in bioreactors, in particular with the aid of organ and tissue-typical mechanical stimuli (e.g.: shearing forces or rhythmical compressions of the cell-laden carrier materials), represents an approach to solving the said problem. The redifferentiation of dedifferentiated cartilage-cells by the application of mechanical stimuli has been described many times.

From DE4306661A1, DE10249903A1, US20060110822A1, DE10208311B4 and US006060306A bioreactor systems are known, in which the cell culture medium moves relative to the culture and thus the exchange of nutrients between the culture and medium is increased (perfusion bioreactors). Partly in said systems the formation of a hyaline cartilage substance (e.g.: collagen II synthesis) is stimulated by the specific use of shearing forces (U.S. Pat. No. 5,928,945A, DE10249903A1, US20050095711A1).

Other bioreactors allow the application of pressure on the cell-laden carrier materials by means of a pressure stamp (WO2005040332A2, DE19808055B4, DE102004012010A1/WO002005087912A3).

In DE102004012010A1/WO002005087912A3 a bioreactor is described which consists of a base plate, a cover plate and a standard multiwell tissue culture plate. Pistons are used in bores of the base plate, which project in the manner of a stamp into the well of the underlying tissue culture plate, leaving a space in which tissue can be cultivated. Said tissue culture chamber is provided with several feed and removal connections for the medium and gassing. An elastic membrane is arranged above the piston, so that a sterile area is formed underneath the membrane. By introducing compressed air the piston can be displaced and thus the cultivated tissue can be compressed.

WO2005040332A2 describes a bioreactor, which consists of a pressure-tight closable reaction chamber, in which a there is a storage area for a tissue construct and a mini-actuator. The reactor chamber is provided with connections for the feed and removal of medium and gassing. Cultivated tissue can be compressed by the displacement of the metal mini-actuator, preferably by the application of a magnetic field. Furthermore, a method is described for producing three-dimensional cell transplants in the aforementioned bioreactor.

The objective of the present invention is to provide a bioreactor for the cultivation of cell and/or tissue cultures with an optimal supply of nutrients.

The objective of the present invention is achieved independently by a bioreactor, wherein at least one distributor plate is arranged in the tissue culture chamber, and a distributor plate with the application of mechanical stimulation and/or perfusion, which comprises recesses, in particular holes, bores, openings and at least one elevation or depression, and a method for cultivating cells and/or tissues in a bioreactor comprising the steps of i) introducing the cells onto a distributor plate in the tissue culture chamber, in particular tissue lumen, ii) supplying the cells with culture medium, iii) mechanical stimulation and/or perfusion. It has proved advantageous that cells or tissue can be differentiated or dedifferentiated cells can be redifferentiated, as by means of the application of mechanical stimuli, such as for example rhythmic compressions, pressure forces, tensile forces, shearing forces etc. are exerted onto the cells of the distributor plate and thus the native system is imitated, such as e.g. cartilage, bone, tendons, ligaments, skin, endothelia, blood vessels, in the body of an individual. By means of perfusion an optimal nutrient supply can be imitated, as is effected in native conditions by a vessel system and is typical of numerous types of tissue, such as e.g. liver tissue and skin. In this way for example autologous tissue can be cultured and prepared for transplantation.

It is also an advantage that the method and the bioreactor can be used for producing large area three-dimensional tissue constructs. The bioreactor according to the invention provides the possibility of medium overflow (for example suitable for the production of cartilage constructs, cartilage bone constructs, tendons, ligaments etc.) and medium throughflow (for example suitable for the production of liver cells or liver equivalents) and/or mechanical stimulation and the combinations of said possibilities with continual process guiding in the closed system. Furthermore, the bioreactor is also suitable for the reconstruction of endothelia and skin transplants, for producing vessel prostheses and the reconstruction of blood vessels. If by means of the bioreactor according to the invention blood vessels or vessel prostheses are produced, to generate the lumen of the vessels preferably an auxiliary structure is used in the bioreactor.

An advantage over the bioreactor described in DE 102004012010A 1 is also the difference from the concept of the stamp projecting into the wells of a tissue culture plate and the introduction of a large area, pressure-applying distributor plate, in order to cultivate large dimensioned tissue transplants, in particular with a diameter of >35 mm.

By providing recesses, in particular holes, bores, openings in the distributor plate it is possible that the cells or the tissue can not only be flowed over with culture medium, but also flowed through or can be flowed over and flowed through, and thus there can be an optimal supply of nutrient to the cells including by diffusion.

It is also the case that the distributor plate comprises at least one elevation or depression, whereby it is ensured that there is a permanent supply of the cells or tissue with culture medium, as even with the exertion of pressure or with compression the medium is not forced completely out of the tissue culture chamber, in particular the medium and residual medium lumen.

Furthermore, the bioreactor in the transitional area from the upper part to the lower part comprises a preferably elastic membrane for dividing into a pressure and tissue culture chamber, whereby there is a combination of elastic and rigid elements and a reactor chamber (tissue culture chamber), which with the presence of various inflows and outflows enables different variants of the medium flow. It has also proved advantageous, that by means of the elasticity of the membrane the pressure exerted by the pressure chamber can be deferred and thus the underlying tissue lumen is compressed and returns back to the initial position.

According to one development at least one cover plate is arranged in the tissue culture chamber, preferably between the membrane and distributor plate, which enables an even transfer of the mechanical stimulation, such as e.g. the pressure exerted by the pressure chamber on the membrane, on the cells or the tissue to be cultured. In addition, the cover plate prevents the collapse of the medium lumen provided that the membrane lies directly on the distributor plate.

Furthermore, the tissue culture chamber comprises at least one inflow and outflow device for the culture medium, which enables the supply of nutrients to the cells or the tissue and medium can be transported back and forth continually.

Furthermore, the tissue culture chamber comprises several lumen, in particular a tissue lumen, a medium lumen and optionally a residual medium lumen, whereby the tissue lumen contains the cells to be cultivated or the cultivated tissue, in the medium lumen, which is for culture medium provided for supplying the cells or the tissue, and in the residual medium lumen the culture medium to be transported away is removed.

In one embodiment variant the tissue lumen is arranged between an upper and a lower distributor plate, whereby the nutrient supply can be optimised via the culture medium.

The inflow device for the culture medium can flow into the medium lumen, whereby the culture medium is distributed evenly by the distributor plate over the cells or the tissue to be cultivated and the biological material can either be flowed over or flowed through.

The outflow device for the culture medium flows out of the residual medium lumen and/or out of the medium lumen, whereby both with the overflowing and through-flowing of the tissue or the cells to be cultivated the continual removal of the culture medium is ensured.

In an advantageous manner the pressure chamber comprises at least one supply device for compressed air, whereby pressure can be exerted via the pressure chamber on the underlying tissue culture chamber.

In a further development it is the case that at least one elastic element is arranged in the tissue culture chamber, in particular in the tissue lumen, which allows compression of the cell and tissue culture and it is also ensured, that the tissue or the cells can extend again after the removal of pressure and the pressure or the shearing force is exerted intermittently or temporarily on the tissue or the cells to be cultivated, as occurs in native tissue such as for example in the cartilage of the knee joint or in blood vessels, in particular endothelia, owing to the pulsing of the blood by heart-muscle contractions. The option of gel polymerisation in the bioreactor—the distributor plate and the elastic element, in particular the elastic ring, are used in this case as a casting mould for the sol—and the omission of an external sowing element increase the user friendliness and the level of integration of the bioreactor system.

Furthermore, it also the case that at least one abutment is arranged in the tissue culture chamber, which determines the maximum compression, whereby excessive compression and possibly damage to the tissue to be cultivated or the cells to be cultivated is prevented.

Between the upper and the lower distributor plate the elastic element and at least one abutment can be arranged, whereby, on the one hand, by means of the elastic element the aforementioned restoring behaviour is ensured and, on the other hand, by means of the abutment the maximum compression is determined.

In a further development, at least one spacer is arranged in the residual medium lumen, whereby it is ensured, on the one hand, that the lower distributor plate is spaced apart from the connection and the circulation of the culture medium can be ensured, and, on the other hand, also a reserve volume is created, in order to increase the variability of the bioreactor, in that the spacer in the residual medium lumen can be removed and thus the tissue lumen can be increased.

In one embodiment variant the upper and lower part are joined together by a detachable connection, whereby in a simple manner the tissue to be cultivated or the cells to be cultivated can be inserted directly into the tissue lumen of the lower part and can be cultivated further after connection to the upper part in sterile conditions.

The lower part can be connected via a detachable connection, in particular a thread, with a stand and/or medium reservoir, whereby a continual supply of culture medium is ensured, or the bioreactor is kept spaced apart from a surface in order for example to ensure greater temperature stability, which otherwise cannot be kept constant by switching off or incubating the bioreactor on a surface, as by means of direct contact over the surface temperature fluctuations can be transferred much more rapidly to the bioreactor. A further advantage is the reliable and user-friendly integration of the medium reservoir into the closed bioreactor system, whereby the bioreactor is secured by means of a thread located in the lower part on a medium bottle, and thus at the same time functions as a medium reservoir and also as a stand for the system, which is thus to be considered as completely closed, and is easy to transport and manipulate.

A culture medium bottle can be used as a stand and/or medium reservoir, whereby a closed bioreactor system consisting of the bioreactor itself and the culture medium bottle can be formed and thus the risk of contamination when supplying the culture medium from an external reservoir is reduced. Furthermore, the medium culture bottle can be autoclaved prior to reuse and the bioreactor simply exchanged.

Furthermore, the inflow and outflow device is connected via tubes to the medium reservoir, whereby a closed connection is formed and in this way also the risk of contamination can be eliminated.

Furthermore, in a further development sensors, in particular flow sensors, dO₂ sensors, pH-sensors, etc. are integrated into the tubes, whereby different parameters can be determined, such as for example the flow volume, the proportion of dissolved oxygen or the pH-value of the culture medium which circulates continually. As soon as a limit value is reached measures can be taken to achieve a defined target value again.

It is the case, that the at least one elevation or depression of the distributor plate is arranged radially, centrally and/or concentrically, whereby the culture medium itself with mechanical stimulation, such as e.g. pressure or pulling, can flow unhindered over or through the distributor plate.

In one embodiment variant several elevations are arranged spaced apart from one another radially in the form of webs, whereby a uniform distribution of the culture medium is made possible over the tissue to be cultivated or the cells to be cultivated, and in addition the collapse of the medium flow caused by the membrane is prevented.

In one development an elevation is arranged in the centre and spaced apart therefrom radial webs are arranged up to the concentric elevation, whereby both flowing over as well as flowing through with culture medium of the tissue to be cultivated is made possible.

In an alternative embodiment an elevation is arranged centrally and at least one further elevation can be arranged radially at the margin, whereby in turn there can be a continual supply of the tissue or cells to be cultivated with culture medium.

Furthermore, at least one channel can be arranged for the supply of culture medium, whereby the supply of culture medium to the distributor plate and thus to the cells to be cultivated and to the tissue to be cultivated can take place.

It is also the case that the culture medium is transported via an inflow device to the tissue culture chamber and leaves the tissue via an outflow device, whereby the supply of culture medium to the tissue to be cultivated or the cells to be cultivated is ensured.

The supply of cells and/or tissue with culture medium can be performed permanently, whereby fresh culture medium can be supplied continually.

The culture medium can either flow through the distributor plate or over the latter or flow through and over the latter, whereby a uniform supply with nutrients is ensured both by diffusion and also perfusion, and thus there can be a uniform growth of the cells to be grown or the tissue to be grown.

For a better understanding of the invention the latter is explained in more detail with reference to the following Figures.

In a much simplified schematic view

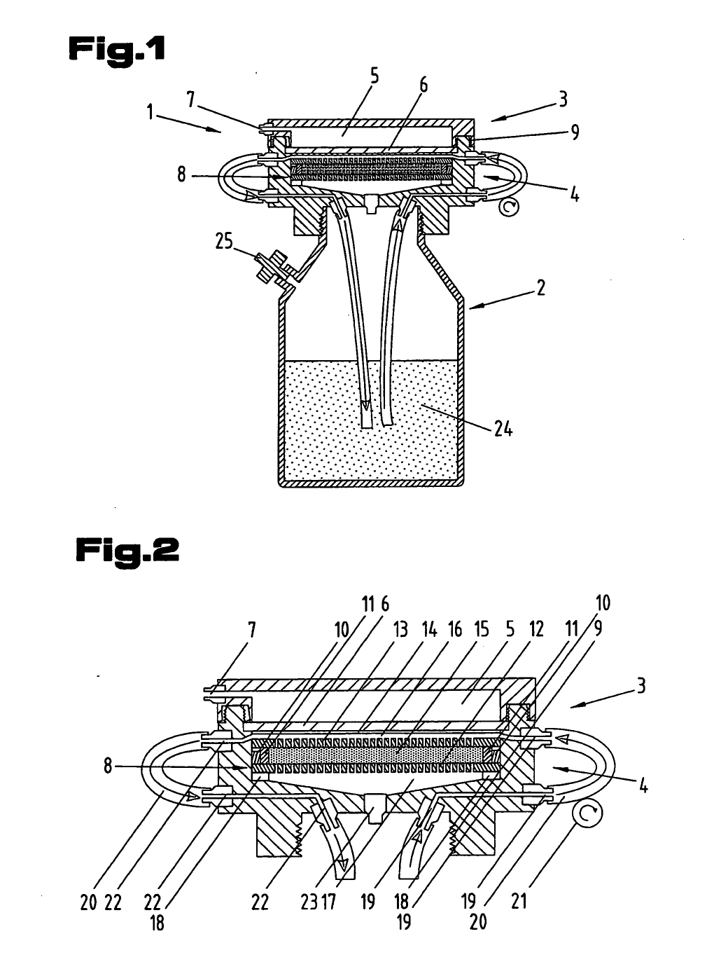

FIG. 1 shows a bioreactor 1 with culture medium bottle 2;

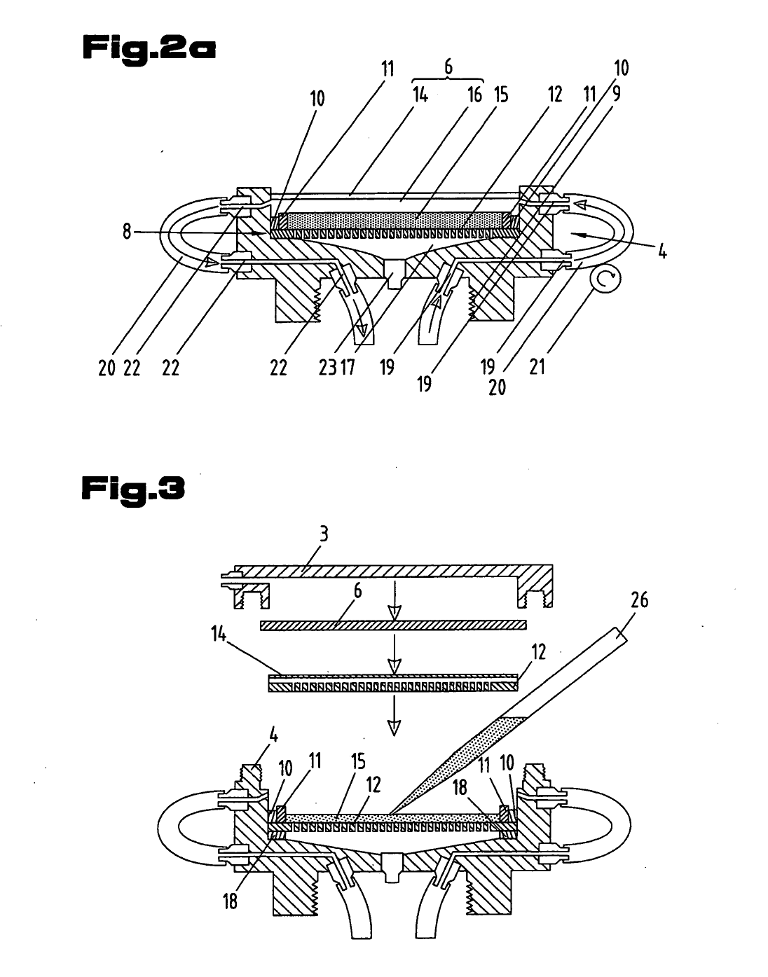

FIG. 2 shows a bioreactor 1;

FIG. 2a shows a further embodiment variant of the bioreactor 1;

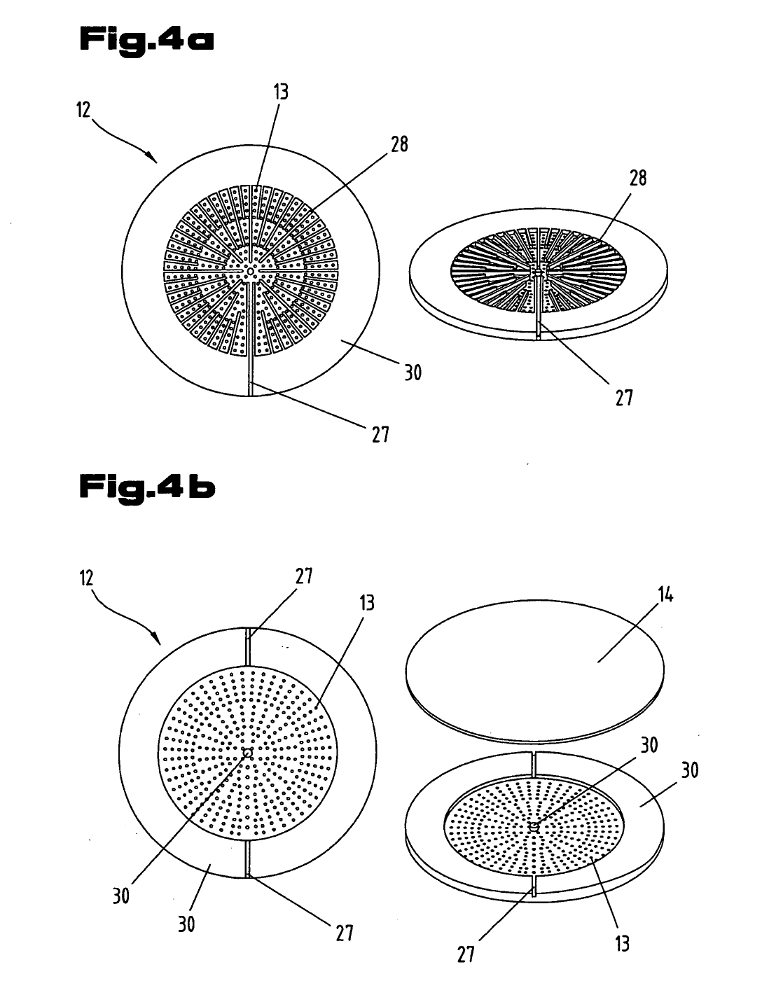

FIG. 3 shows an exploded view of a bioreactor 1;

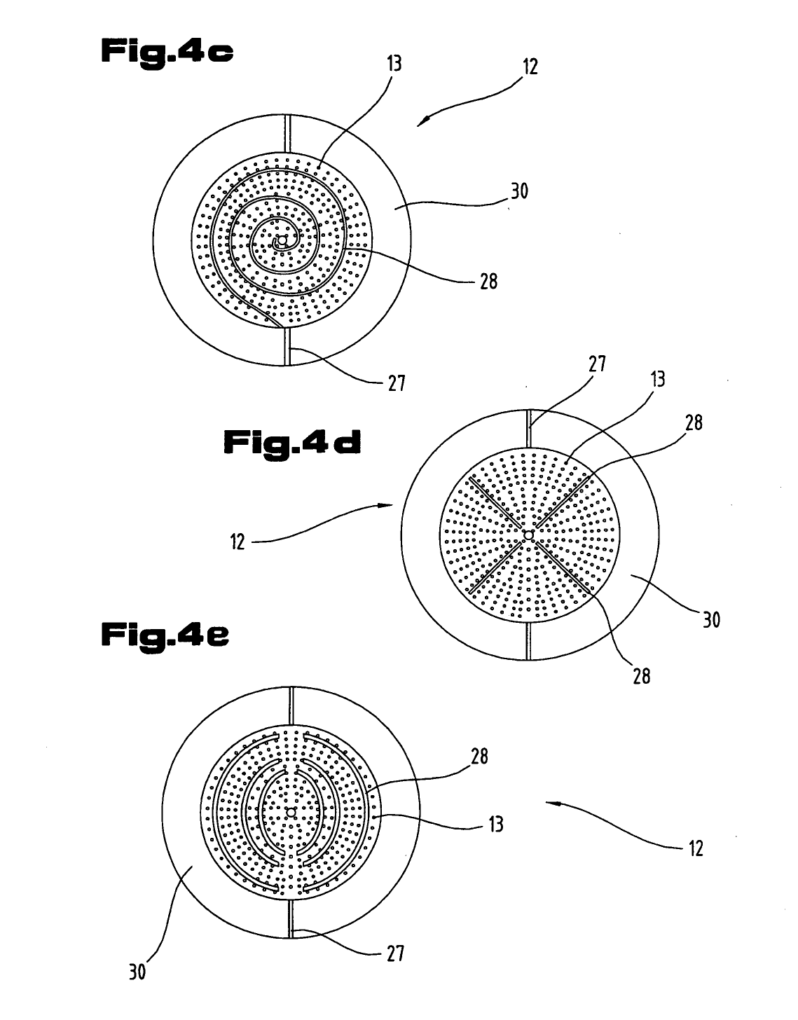

FIG. 4 a, b, c, d and e show various different embodiment variants of a distributor plate 12.

First of all, it should be noted that in the variously described exemplary embodiments the same parts have been given the same reference numerals and the same component names, whereby the disclosures contained throughout the entire description can be applied to the same parts with the same reference numerals and same component names. Also details relating to position used in the description, such as e.g. top, bottom, side etc. relate to the currently described and represented figure and in case of a change in position should be adjusted to the new position. Furthermore, also individual features or combinations of features from the various exemplary embodiments shown and described can represent in themselves independent or inventive solutions.

All of the details relating to value ranges in the present description are defined such that the latter include any and all part ranges, e.g. a range of 1 to 10 means that all part ranges, starting from the lower limit of 1 to the upper limit 10 are included, i.e. the whole part range beginning with a lower limit of 1 or above and ending at an upper limit of 10 or less, e.g. 1 to 1.7, or 3.2 to 8.1 or 5.5 to 10.

The present invention relates to a bioreactor 1 for the cultivation and growth of cells or tissues with simultaneous mechanical stimulation and/or perfusion. The mechanical stimulation can be performed by pressure, pulling or shearing forces. Preferably, in the bioreactor 1 three-dimensional cell and tissue cultures are grown for tissue reconstruction. The described bioreactor 1 can be used for producing various different types of artificial tissue, for example for producing artificial cartilage for autologous cartilage transplantation. Additional applications are bone or cartilage-bone transplants, tendons, ligaments, skin, vessels, etc.

Various different embodiment variants of the bioreactor according to the invention

FIG. 1 shows an embodiment variant of the bioreactor 1 with a culture medium bottle 2 for tissue reconstruction in a closed state.

Said embodiment variant of the bioreactor 1 consists of an upper part 3 and a lower part 4. The upper part 3 surrounds a pressure chamber 5, which is delimited on one side by an elastic membrane 6 and can be pressurised with compressed air through an opening 7. The lower part 4 surrounds a tissue culture chamber 8. It is connected to the upper part 3, preferably by a thread 9, whereby the elastic membrane 6 is clamped and the pressure chamber 5 is closed tightly against the tissue culture chamber 8 enclosed by the lower part 4.

FIG. 2 shows further details of the first embodiment variant of the bioreactor 1. Said embodiment variant of the bioreactor 1 also comprises an abutment 10, for example an upper spacer, in order to determine the maximum compression in the tissue culture chamber 8. An elastic element 11, in particular an elastic ring or a spring, ensures the return to the starting position.

Furthermore, at least one distributor plate 12, preferably with recesses 13, is provided. The distributor plate 12 can e.g. comprise a perforated base. Between the distributor plate 12 and the cover plate 14 the tissue lumen 15 is arranged.

Furthermore, the tissue culture chamber 8 comprises a medium lumen 16 next to the tissue lumen 15, where the tissue to be cultivated or the cells to be cultivated are established, where culture medium is loaded, and optionally a residual medium lumen 17, where culture medium is transported away.

In the embodiment, which is shown in FIG. 2, the tissue lumen 15 is formed between an upper and a lower distributor plate 12. The residual medium lumen 17 is arranged adjacent to the tissue lumen 15.

For spacing the lower distributor plate 12 from the base of the residual medium lumen 17 a spacer 18 is provided.

Between the upper distributor plate 12 and the cover plate 14 the medium lumen 16 is arranged into which the culture medium enters.

A system of feed channels 19, which is closed by a tube 20, and a tube pump 21, enable the entry of culture medium into the tissue culture chamber 8 of the lower part 4, in particular into the medium lumen 16 adjacent to the upper distributor plate 12.

A system of removing channels 22, closed by a tube 20, enables the outflow of culture medium from the residual medium lumen 17 of the lower part 4. The exchange (diffusion) of nutrients and metabolites between the culture medium flowing through the medium lumen 16 of the upper distributor plate 12 and tissue located in the tissue lumen 15 is made possible through openings 7 in the base of the upper distributor plate 12.

Alternatively to the variant of overflowing with medium, the tissue located in the tissue lumen 15 can be flowed through by culture medium. For this type of operation of the bioreactor 1 the medium-removing channels 22 are closed by blind closures, and the connection 23 for medium removal is opened. Also switching between both operating modes (flowing over and through) with continual process guiding is possible and can be used for tissue, the mechanical properties (e.g.: flowability) of which change during maturation in the cell culture.

The shown embodiment variant makes it possible to screw the bioreactor 1 by means of a thread 9 (e.g.: GL45-thread) onto a culture medium bottle 2. The culture medium bottle 2 is thus used as a medium reservoir and as a standing foot of the closed system. The medium supplying and removing channels 19, 22 of the bioreactor 1 can be connected to tubes 20, which dip into the medium reservoir 24 of the culture medium bottle 2. A gas-permeable filter, in particular a sterile filter 25, can be used for the exchange of gas between the closed bioreactor system and the surrounding atmosphere (for example: inner chamber of a cell culture incubator). By means of continual operation as a closed system and the thereby connected securing of sterile tissue culture conditions the bioreactor 1 meets the requirements of the WHO Guidelines for Good Manufacturing Practice. The tube connections in the system of the medium supplying and removing channels 19, 22 can be used for the integration of sensors (for example: flow sensors, dO2 sensors, pH sensors) whilst maintaining the closed nature of the medium circuit in the bioreactor.

The shown embodiment variant of the bioreactor enables the application of pressure pulses and shearing forces onto the tissue cultivated in the tissue culture chamber 8, in particular in the tissue lumen 15. For this by introducing compressed air into the pressure chamber 5 overpressure can be generated, which is transferred by the elastic membrane 6 via the cover plate 14 onto the upper distributor plate 12 and transferred from the latter onto the tissue located in the tissue culture chamber 8. The bearing of the upper distributor plate 12 on an elastic element 11, in particular ring, enables its movement downwards, towards the cultivated tissue, but only so far that it bears on the solid upper abutment 10 functioning as a stop, in particular the upper spacer. It is not the strength of the overpressure (for example: 0.2-0.5 Bar), but rather the height difference between the elastic ring and upper spacer that determines precisely to what degree the cultivated tissue is compressed. The shown embodiment variant of the bioreactor 1 allows the application of pressure pulses and shearing forces without interrupting the medium flow.

In an alternative embodiment variant shown in FIG. 2a, the tissue to be grown or the cells to be cultivated can also be arranged on a distributor plate 12 and not as shown in FIGS. 1 and 2 between two distributor plates 12. Thus for example the tissue lumen 15 can join directly to the medium lumen 16 and be delimited by a cover plate 14. The arrangement of the pressure chamber 5 is omitted, as, for example by means of a stamp, intermittent pressure can be exerted on the cover plate 14 and this transferred to the tissue or the cells in the tissue lumen 15. The mobility of the cover plate 14 can for example be achieved by a seal made of elastic material in the connection area with the bioreactor 1. By means of exerting pressure also shearing forces act automatically on the cells.

Furthermore, FIG. 2a shows an embodiment variant without a lower spacer 18 under the distributor plate 12 in the residual medium lumen 17. By removing said spacer 18 the tissue lumen 15 can be increased and the residual medium lumen 17 can be made smaller.

FIG. 3 shows an embodiment variant of the bioreactor for tissue reconstruction in the opened state during the introduction of a cell-loaded collagen I sol into the bioreactor 1. Of course, the cells can also be introduced without carriers, i.e. in suspension or on other carriers.

Into the sterile lower part 4 of the bioreactor 1 provided with a sterile lower spacer 18, a sterile lower distributor plate 12, a sterile abutment 10, in particular upper spacer and a sterile elastic element 11, such as e.g. a ring, a cell-laden collagen I-sol can be inserted with a pipette 26 whilst keeping to the sterile conditions. After the transfer of the collagen I-sol into the gel state it fills out the entire tissue lumen 15. The tissue culture chamber 8, in particular the tissue lumen 15, can be sealed tightly by placing a sterile upper distributor plate 12 and a sterile elastic membrane 6 and by screwing on the bioreactor upper part 3.

Other examples of application are the introduction of cell-loaded fleece and fibre materials, cell-loaded porous structures and cell-loaded composites of gel, fleece, fibre-like and/or porous carrier materials into the tissue culture chamber 8.

By varying the dimensions of the abutment 10, in particular of the upper spacer, the elastic element 11, such as the ring and the lower spacer 18, by using the volume in the residual medium lumen 17, both the thickness and diameter of the cultivated tissue and also the strength of the possibly applied mechanical stimulation, e.g. compression, shearing force, etc., can be adjusted.

FIGS. 4a-e show embodiment variants of the upper distributor plate 12 of the bioreactor 1 according to the invention.

In the first variant in FIG. 4a inflowing culture medium can be taken up through a channel 27 and released downwards through the recesses 13 in the base. Webs 28 are arranged radially in order to achieve an even distribution of the culture medium. It is possible for the tissue cultivated on the distributor plate 12 or between the distributors plate 12 to be flowed through. In variant a the closely arranged webs 28 prevent the collapse of the medium through-flowed medium lumen 16 of the distributor plate 12 during mechanical stimulation, e.g. during the pressure pulse. The webs 28 also prevent the elastic membrane 6 from bearing closely against the distributor plate 12 or the tissue located thereon on the exertion of mechanical stimulation and thus closes the recesses 13, which would interrupt the supply of culture medium.

In variant b of the distributor plate 12, as shown in FIG. 4b, culture medium can be taken up through a channel 27 and released again by a second channel 27. Nutrient and metabolite diffusion between the overflowing culture medium and the tissue under the plate is made possible through recesses 13 in the base of the plate. Both variant a and variant b can receive pressure pulses and transfer them to the underlying tissue. In variant b in FIG. 4b this function is taken over by a solid disc 29 with bearing points on the edge and in the centre, i.e. the elevations 30, of the distributor plate 12.

A third embodiment variant c of the distributor plate 12 is shown in FIG. 4c. The elevations 30 are arranged in a spiral. In addition, a central elevation 30 is shown, whereby the distributor plate 12 also functions without said elevation 30, as the spiralling webs 28 can perform its function.

FIG. 4d shows a further embodiment variant of the distributor plate 12, wherein optionally one elevation 30 is arranged centrally and individual webs 28 are spaced apart therefrom.

FIG. 4e shows a laminar arrangement of the elevation 30 on a distributor plate 12. The elevations can be arranged evenly over the distributor plate 12 or more closely together in the edge area and less closely together near the centre.

Further embodiment variants of the distributor plate 12 are conceivable, whereby the latter guide the medium flow over and/or through the cultivated tissue, and at the same can transfer a mechanical stimulation, such as pressure pulses, tensile and/or shearing forces onto the cultivated tissue.

The exemplary embodiments show possible embodiment variants of the bioreactor 1 and the distributor plate 12, whereby it should be noted at this point that the invention is not restricted to the embodiment variants shown in particular, but rather various different combinations of the individual embodiment variants are also possible and this variability, due to the teaching on technical procedure, lies within the ability of a person skilled in the art in this technical field. Thus all conceivable embodiment variants, which are made possible by combining individual details of the embodiment variants shown and described, are also covered by the scope of protection.

### Areas of Application

Areas of application of the bioreactor 1 according to the invention are:

(1) cartilage reconstruction for autologous cartilage transplantation (ACT),
(2) reconstruction of cartilage for research purposes,
(3) the production of cartilage-bone-constructs for transplantation in cartilage-bone-defects,
(4) the production of cartilage-bone-constructs for research purposes,
(5) the production of three-dimensional liver equivalents for toxicological, pharmaceutical and biological examinations,
(6) the production and reconstruction of ligaments, tendons, etc.,
(7) the production and reconstruction of intervertebral discs, in particular cells of the annulus fibrosus,
(8) the production and reconstruction of skin transplants,
(9) the production and reconstruction of blood vessels, in particular endothelia, for vessel prostheses and vessel transplants, and
(10) other applications from the field of tissue engineering with the aim of reconstructing a three-dimensional tissue.

### Example of Application 1

In the first example of an application primary chondrocytes are harvested from the knee cartilage of a young pig and cultivated for 14 days in a 3% collagen I-gel in DMEM/Ham's F12-medium with 50 μg/ml ascorbate-2-phosphate and 10% FCS in a bioreactor. Culture medium flows over throughout the cultivation. After the cultivation collagen II and aggrecan are verified immuncytochemically. Both proteins are marker proteins for hyaline cartilage. A portion of the tissue constructs is subjected after one week of unloaded cultivation to a one-week mechanical loading program. The unloaded culture has low immunity reactivity to collagen II (collagen II red, cell nuclei blue) and aggrecan (aggrecan red, cell nuclei blue). However, the compression promotes the formation of collagen II (collagen II red, cell nuclei blue) and aggrecan (aggrecan red, cell nuclei blue). In the bioreactor 1 with mechanical loading accordingly a cartilage construct can be generated, the extracellular matrix of which corresponds to that of hyaline cartilage. The dedifferentiation of the primary chondrocytes is counteracted.

### Example of Application 2

In the second example of an application primary chondrocytes are harvested from the knee cartilage of a young pig, dedifferentiated in three day monolayer-culture and then cultivated in a bioreactor for three weeks in a 3% collagen I-gel in DMEM/Ham's F12 medium with 50 μg/ml ascorbate-2-phosphate and 10% FCS. Throughout the duration of the culture the collagen gel is flowed over with culture medium and compressed rhythmically in an interval of 12 hours for two hours at a frequency of 0.5 Hz. In the control experiment a collagen gel is cultivated for three weeks, flowed over with medium, but not exposed to any mechanical compression. After the cultivation collagen II and aggrecan are verified immuncytochemically. Both proteins are marker proteins for hyaline cartilage. The unloaded culture of the control experiment shows low immune reactivity to collagen II and aggrecan. However, compression encourages the formation of collagen II and aggrecan. By means of mechanical stimulation in the bioreactor thus dedifferentiated primary chondrocytes can be redifferentiated and form features of a hyaline cartilage.

FIGS. 4b, 4c, 4d and 4e show an additional and optionally independent embodiment of the distributor plate 12, wherein for the same parts the same reference numerals and component names are used as in the preceding FIG. 4a. To avoid unnecessary repetition, reference is made to the detailed description in the preceding Figs.

Finally, as a point of formality, it should be noted that for a better understanding of the structure of the bioreactor 1 and the distributor plate 12, the latter and its components have not been represented true to scale in part and/or have been enlarged and/or reduced in size.

The underlying objective of the independent solutions according to the invention can be taken from the description.

Mainly the individual embodiments shown in FIGS. 1, 2, 2a, 3, 4a-e can form the subject matter of independent solutions according to the invention. The objectives and solutions according to the invention relating thereto can be taken from the detailed descriptions of these figures.

### LIST OF REFERENCE NUMERALS

1 Bioreactor
2 Culture medium bottle
3 Upper part
4 Lower part
5 Pressure chamber
6 Membrane
7 Opening
8 Tissue culture chamber
9 Thread
10 Abutment
11 Elastic element
12 Distributor plate
13 Recess
14 Cover plate
15 Tissue lumen
16 Medium lumen
17 Residual medium lumen
18 Spacer
19 Channel
20 Tube
21 Pump
22 Channel
23 Connection
24 Medium reservoir
25 Filter
26 Pipette
27 Channel
28 Web
29 Disc
30 Elevation

1. Bioreactor (1) for cell and tissue culture for the mechanical stimulation and/or perfusion of tissue or cell cultures, comprising an upper part (3) and a lower part (4) connected thereto, with a tissue culture chamber (8), wherein in the tissue culture chamber (8) at least one distributor plate (12) is arranged.

2. Bioreactor (1) according to claim 1, wherein the distributor plate (12) comprises recesses (13), in particular holes, bores, openings.

3. Bioreactor (1) according to claim 1, wherein the distributor plate (12) comprises at least one elevation (30) or depression.

4. Bioreactor (1) according to claim 1, wherein in the transitional area from the upper part (3) to the lower part (4) a membrane (6) is arranged for dividing into a pressure chamber (5) and the tissue culture chamber (8).

5. Bioreactor (1) according to claim 1, wherein the membrane (6) is elastic.

6. Bioreactor (1) according to claim 1, wherein the upper part (3) comprises at least one cover plate (14).

7. Bioreactor (1) according to claim 1, wherein the at least one cover plate (14) is arranged in the tissue culture chamber (8) between the membrane (6) and distributor plate (12).

8. Bioreactor (1) according to claim 1, wherein the tissue culture chamber (8) comprises at least one inflow and outflow device, in particular an inflowing (19) and outflowing channel (22), for a culture medium.

9. Bioreactor (1) according to claim 1, wherein the tissue culture chamber (8) comprises several lumen, in particular a tissue lumen (15), a medium lumen (16) and if necessary a residual medium lumen (17).

10. Bioreactor (1) according to claim 1, wherein the tissue lumen (15) is arranged between an upper and a lower distributor plate (12).

11. Bioreactor (1) according to claim 1, wherein the inflow device for the culture medium flows into the medium lumen (16).

12. Bioreactor (1) according to claim 1, wherein the outflow device for the culture medium flows from the residual medium lumen (17) and/or medium lumen (16).

13. Bioreactor (1) according to claim 1, wherein the pressure chamber (5) comprises at least one feed device for compressed air.

14. Bioreactor (1) according to claim 1, wherein at least one elastic element (11) is arranged in the tissue culture chamber (8), in particular in the tissue lumen (15), which allows the compression of the cell and tissue culture.

15. Bioreactor (1) according to claim 1, wherein at least one abutment (10) is arranged in the tissue culture chamber (8), which determines the maximum compression.

16. Bioreactor (1) according to claim 1, wherein between the upper and lower distributor plate (12) the elastic element (11) and the abutment (10) are arranged.

17. Bioreactor (1) according to claim 1, wherein at least one spacer (18) is arranged in the residual medium lumen (17).

18. Bioreactor (1) according to claim 1, wherein the upper part and the lower part (3, 4) are joined together by a detachable connection.

19. Bioreactor (1) according to claim 1, wherein the lower part (4) is connected via a detachable connection, in particular a thread (9), with a support and/or medium reservoir (24).

20. Bioreactor (1) according to claim 19, wherein a culture medium bottle (2) forms the support and/or medium reservoir (24).

21. Bioreactor (1) according to claim 1, wherein the inflow and outflow devices are connected by tubes (20) to the medium reservoir (24).

22. Bioreactor (1) according to claim 1, wherein sensors, in particular flow sensors, dO₂-sensors, pH-value sensors, are integrated into the tubes (20).

23. Use of the bioreactor (1) according to claim 1 for the culture of cell and tissue culture for tissue reconstruction.

24. Distributor plate (12) for cell and tissue culture, in particular with the use of mechanical stimulation and/or perfusion, wherein recesses (13), in particular holes, bores, openings and at least one elevation (30) or depression are provided.

25. Distributor plate (12) according to claim 24, wherein the at least one elevation (30) is arranged radially, centrally and/or concentrically.

26. Distributor plate (12) according to claim 24, wherein several elevations (30) are arranged spaced apart radially from one another in the form of webs (28).

27. Distributor plate (12) according to claim 24, wherein an elevation (30) is arranged centrally and spaced apart from the latter radial webs (28) are arranged up to the concentric elevation (30).

28. Distributor plate (12) according to claim 24, wherein an elevation (30) is arranged centrally, and at least one further elevation (30) is arranged radially on the margin.

29. Distributor plate (12) according to claim 24, wherein at least one channel (27) is provided for a culture medium.

30. Method for cultivating cells and/or tissue in a bioreactor (1), according to claim 1, comprising the steps i) introducing the cells onto a distributor plate (12) into a tissue culture chamber (8), in particular tissue lumen (15), ii) supplying the cells with culture medium, iii) mechanical stimulation and/or perfusion of the cells or tissue culture cells.

31. Method according to claim 30, wherein the culture medium is transported via an inflow device, in particular a feed channel (19), to the tissue culture chamber (8), in particular the tissue lumen (15), and via an outflow device, in particular a removing channel (22), leaves the tissue culture chamber (8), in particular the tissue lumen (15).

32. Method according to claim 30, wherein the cells and/or tissue are provided permanently with culture medium.

33. Method according to claim 30, wherein the culture medium flows through and/or flows over the distributor plate (12).
